(19) United States
(12) Patent Application Publication
(10) Pub. No.: US 2025/0281098 A1
(43) Pub. Date: Sep. 11, 2025

(54) ELECTROCARDIOGRAPHY PROCESSING DEVICE

(71) Applicant: Quanta Computer Inc., Taoyuan City (TW)

(72) Inventors: Jia-Li SUNG, Taoyuan City (TW); Yung-Ming CHUNG, Taoyuan City (TW)

(21) Appl. No.: 18/746,556

(22) Filed: Jun. 18, 2024

(30) Foreign Application Priority Data
Mar. 5, 2024 (TW) 113107815

(51) Int. Cl. A61B 5/352; A61B 5/00; A61B 5/35

(52) U.S. Cl. CPC A61B 5/352; A61B 5/35; A61B 5/726; A61B 5/7267

(57) ABSTRACT

An electrocardiography processing device includes a measurement unit and a processing unit. The measurement unit measures an object to generate an electrocardiography signal. The processing unit receives the electrocardiography signal, classifies the electrocardiography signal using a first classification model to classify the electrocardiography signal into a first type or a second type, and classifies the electrocardiography signal of the first type using a second classification model to classify the electrocardiography signal of the first type into the first type or the second type.

### CROSS REFERENCE TO RELATED APPLICATIONS

This application claims priority of Taiwan Patent Application No. 113107815, filed on Mar. 5, 2024, the entirety of which is incorporated by reference herein.

### BACKGROUND OF THE INVENTION

### Field of the Invention

The present invention relates to a processing device, and in particular it relates to an electrocardiography processing device.

### Description of the Related Art

In general, portable electrocardiogram measurement equipment may measure electrocardiography (ECG) waveforms very conveniently and store real-time waveforms using mobile phone applications (APPs) for reference by medical personnel.

However, the analysis of electrocardiography waveforms is relatively time-consuming and subjective for medical personnel, and a large amount of electrocardiography data may also bring additional pressure to medical personnel and may cause misjudgments and reduce the accuracy of electrocardiography data analysis. Therefore, how to effectively increase the accuracy of electrocardiography data analysis has become a focus for technical improvements by various manufacturers.

### BRIEF SUMMARY OF THE INVENTION

An embodiment of the present invention provides an electrocardiography processing device, thereby effectively increasing the accuracy of the type classification of the electrocardiography signal.

An embodiment of the present invention provides an electrocardiography processing device, which includes a measurement unit and a processing unit. The measurement unit is configured to measure an object to generate an electrocardiography signal. The processing unit is configured to receive the electrocardiography signal, classify the electrocardiography signal using a first classification model to classify the electrocardiography signal into a first type or a second type, and classify the electrocardiography signal of the first type using a second classification model to classify the electrocardiography signal of the first type into the first type or the second type.

According to the electrocardiography processing device disclosed by the present invention, the processing unit receives the electrocardiography signal generated by the measurement unit, classifies the electrocardiography signal using the first classification model to classify the electrocardiography signal into the first type or the second type, and classifies the electrocardiography signal of the first type using the second classification model to classify the electrocardiography signal of the first type into the first type or the second type. Therefore, the accuracy of the type classification of the electrocardiography signal may be effectively increased.

### BRIEF DESCRIPTION OF THE DRAWINGS

The present invention can be more fully understood by reading the subsequent detailed description and examples with references made to the accompanying drawings, wherein:

FIG. 1 is a schematic view of an electrocardiography processing device according to an embodiment of the present invention;

FIG. 2 is a schematic view of an operation of an electrocardiography processing device according to an embodiment of the present invention;

FIG. 3 is a schematic view of an operation of an electrocardiography processing device according to an embodiment of the present invention;

FIG. 4 is a schematic view of an operation of an electrocardiography processing device according to an embodiment of the present invention;

FIG. 5 is a schematic view of R-wave interval segments, (m−1)-th dimensional sub R-wave interval segment sequence, m-th dimensional sub R-wave interval segment sequence and (m+1)-th dimensional sub R-wave interval segment sequence according to an embodiment of the present invention;

FIG. 6A and FIG. 6B are a schematic view of a processing unit calculating the A value according to an embodiment of the present invention;

FIG. 6C and FIG. 6D are a schematic view of a processing unit calculating the B value according to an embodiment of the present invention;

FIG. 7 is a flowchart of an operation method of an electrocardiography processing device according to an embodiment of the present invention;

FIG. 8 is a detailed flowchart of step S706 in FIG. 7;

FIG. 9 is a detailed flowchart of step S708 in FIG. 7; and

FIG. 10 is a detailed flowchart of step S908 in FIG. 9.

### DETAILED DESCRIPTION OF THE INVENTION

In each of the following embodiments, the same reference number represents an element or component that is the same or similar.

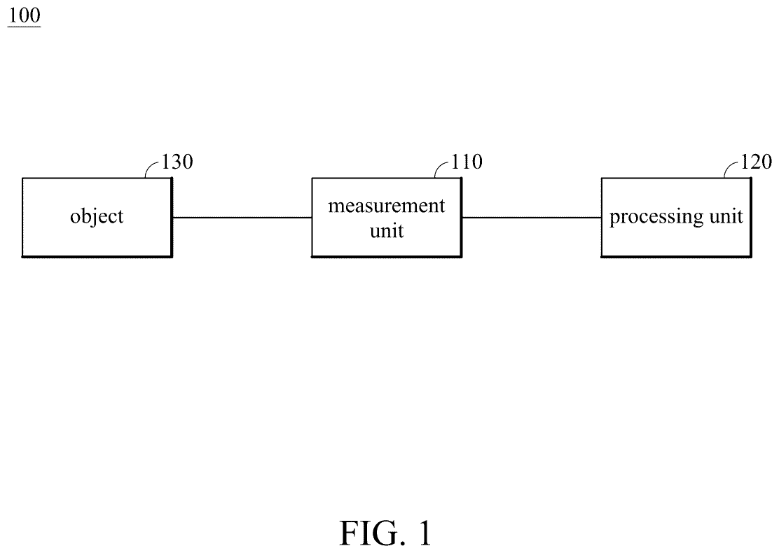

FIG. 1 is a schematic view of an electrocardiography processing device according to an embodiment of the present invention. Please refer to FIG. 1. The electrocardiography processing device 100 may include a measurement unit 110 and a processing unit 120.

The measurement unit 110 measures an object 130 to generate an electrocardiography (ECG) signal. In the embodiment, the measurement unit 110 has, for example, measurement electrodes, and the measurement electrodes may be in contact with the object 130 in order to measure the electrocardiogram of the object 130 to generate the electrocardiography signal.

The processing unit 120 may be coupled to the measurement unit 110. The processing unit 120 may receive the electrocardiography signal generated by the measurement unit 110. Then, the processing unit 120 may classify the electrocardiography signal using a first classification model to classify the electrocardiography signal into a first type or a second type. In the embodiment, the first type is, for example, an arrhythmic type, and the second type is, for example, a non-arrhythmic type. In addition, the arrhythmic type includes an atrial fibrillation (AF) and an atrial flutter (AFL).

Furthermore, the processing unit 120 may obtain a training data set. For example, the processing unit 120 may obtain the training data set from the database. In addition, the training data set includes, for example, a known electrocardiography signal of a time length, and the known electrocardiography signal may include the first type (the arrhythmic type) and the second type (the non-arrhythmic type). Furthermore, the above time length is, for example, several minutes or hours, but the embodiment of the present invention is not limited thereto.

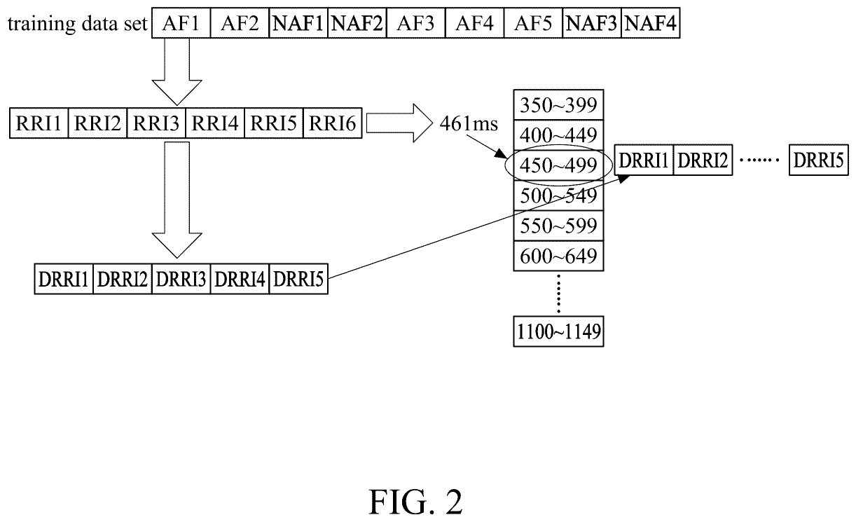

Then, the processing unit 120 may use a predetermined time length to divide the training data set into a plurality of first signal segments AF1˜AF5 and a plurality of second signal segments NAF1˜NAF4, as shown in FIG. 2. In some embodiments, the above predetermined time length is, for example, 30 seconds, but the embodiment of the present invention is not limited thereto. The user may adjust the above predetermined time length according to the requirements thereof.

In the embodiments, the type of the first signal segments AF1˜AF5 is different from the type of the second signal segments NAF1˜NAF4. For example, the type of the first signal segments AF1˜AF5 is the first type (the arrhythmia type), and the type of the second signal segments NAF1˜NAF4 is the second type (the non-arrhythmia type).

In addition, the first signal segments AF1˜AF5 and the second signal segments NAF1˜NAF4 do not overlap each other. For example, the first signal segment AF1 is 1˜30 seconds, the first signal segment AF2 is 31˜60 seconds, the second signal segment NAF1 is 61˜90 seconds, the second signal segment NAF2 is 91˜120 seconds, the first signal segment AF3 is 121˜150 seconds, the first signal segment AF4 is 151˜180 seconds, the first signal segment AF5 is 181˜210 seconds, the second signal segment NAF3 is 211˜240 seconds, and the second signal segment NAF4 is 241˜270 seconds.

Afterward, the processing unit 120 may perform an R-wave interval (R-R interval) detection on each of the first signal segments AF1˜AF5 to obtain a plurality of R-wave interval segments of each of the first signal segments AF1˜AF5. For example, after the processing unit 120 performs the R-wave interval detection on the first signal segment AF1, the processing unit 120 may obtain six R-wave interval segments of the first signal segments AF1, such as RRI1, RRI2, RRI3, RRI4, RRI5 and RRI6. The R-wave interval segments of the rest of the first signal segments AF2˜AF5 may be deduced by analogy.

Then, the processing unit 120 may perform an R-wave interval average value calculation on the R-wave interval segments of each of the first signal segments AF1˜AF5 to obtain an R-wave interval average value of each of the first signal segments AF1˜AF5. For example, the processing unit 120 may perform the R-wave interval average value calculation on the R-wave interval segments RRI1, RRI2, RRI3, RRI4, RRI5 and RRI6 of the first signal segment to the R-wave interval average value of the first signal segment AF1, such as 461 milliseconds (ms). The R-wave interval average values of the rest of the first signal segments AF2˜AF5 may be deduced by analogy.

Afterward, the processing unit 120 sets a plurality of interval templates, and the above interval templates may include different time ranges. For example, the processing unit 120 may set 16 interval templates, wherein the first interval template may have a time range of 350˜399 milliseconds, the second interval template may have a time range of 400˜449 milliseconds, the third interval template may have a time range of 450˜499 milliseconds, the fourth interval template may have a time range of 500˜549 milliseconds, the fifth interval template may have a time range of 550˜599 milliseconds, the sixth interval template may have a time range of 600˜649 milliseconds, . . . , the sixteenth interval template may have a time range of 1100˜1149 milliseconds, but the embodiment of the present invention is not limited thereto.

Then, the processing unit 120 may distribute the first signal segments AF1˜AF5 to the corresponding interval templates according to the R-wave interval average value of the first signal segments AF1˜AF5. For example, the processing unit 120 may distribute the first signal segment AF1 to the corresponding interval template (such as the third interval template (450˜499 milliseconds)) according to the R-wave interval average value of the first signal segment AF1 being 461 milliseconds. The allocation of the rest of the first signal segments AF2˜AF5 may be deduced by analogy.

Afterward, the processing unit 120 may perform an R-wave interval difference value calculation on each of the first signal segments AF1˜AF5 to obtain a plurality of R-wave interval difference values of each of the first signal segments AF1˜AF5. That is, the processing unit 120 may perform the R-wave interval difference value calculation on the R-wave interval RRI1, RRI2, RRI3, RRI4, RRI5 and RRI6 of the first signal segment AF1 to obtain the R-wave interval difference values DRRI1, DRRI2, DRRI3, DRRI4 and DRRI5 of the first signal segment AF1.

In the embodiment, the R-wave interval difference value DRRI1 is the R-wave interval RRI2 minus the R-wave interval RRI1, the R-wave interval difference value DRRI2 is the R-wave interval RRI3 minus the R-wave interval RRI2, the R-wave interval difference value DRRI3 is the R-wave interval RRI4 minus the R-wave interval RRI3, the R-wave interval difference value DRRI4 is the R-wave interval RRI5 minus the R-wave interval RRI4, and the R-wave interval difference value DRRI5 is the R-wave interval RRI6 minus the R-wave interval RRI5. The calculation of the R-wave interval difference values of the rest of the first signal segments AF2˜AF5 may be deduced by analogy.

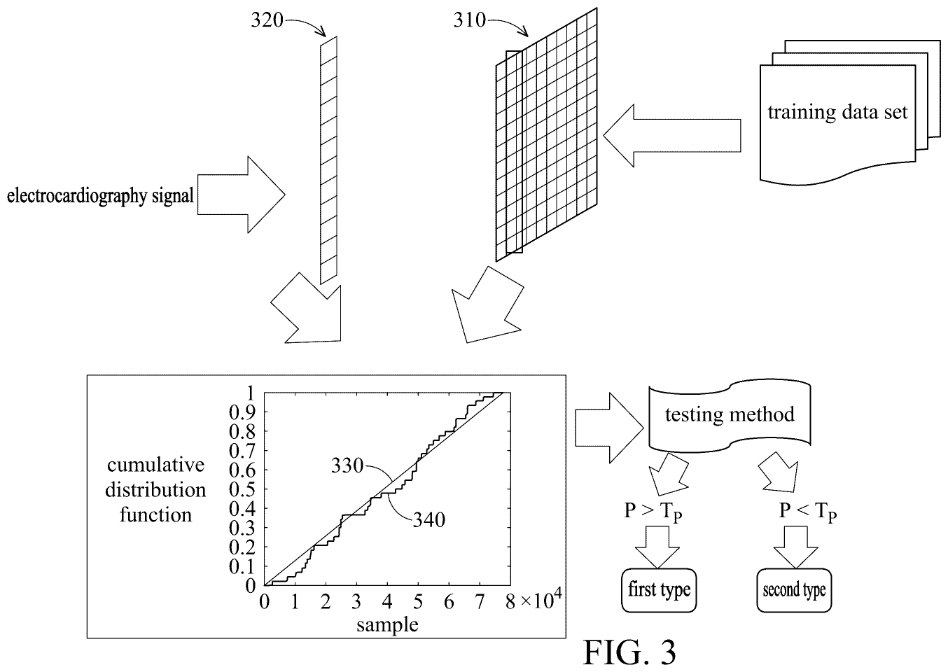

Then, the processing unit 120 may distribute the R-wave interval difference values of the first signal segments AF1˜AF5 to the interval templates corresponding to the first signal segments AF1˜AF5 to generate the first classification model 310, as shown in FIG. 3. For example, the processing unit 120 may distribute the R-wave interval difference values DRRI1, DRRI2, DRRI3, DRRI4 and DRRI5 of the first signal segment AF1 to the interval template corresponding to the first signal segment AF1 (such as the third interval template (450˜499 milliseconds)). The allocation of the R-wave interval difference values of the rest of the first signal segments AF2˜AF5 may be deduced by analogy.

Afterward, the processing unit 120 may perform the R-wave interval average value calculation on the electrocardiography signal to obtain the R-wave interval average value of the electrocardiography signal. In the embodiment, the R-wave interval average value of the electrocardiography signal is, for example, 460 milliseconds, but the embodiment of the present invention is not limited thereto.

Then, the processing unit 120 may select a first interval template of the interval templates in the first classification model 310 according to the R-wave interval average value of the electrocardiography signal. For example, the processing unit 120 may select first interval template (such as the third interval template (such as 450˜499 milliseconds)) corresponding to the R-wave interval average value (such as 460 milliseconds) from the interval templates in the first classification model 310 according to the R-wave interval average value (such as 460 milliseconds) of the electrocardiography signal.

Afterward, the processing unit 120 may perform the R-wave interval difference value calculation on the electrocardiography signal to obtain a plurality of R-wave interval difference values 320 of the electrocardiography signal. Then, the processing unit 120 may perform a cumulative distribution function calculation on the R-wave interval difference values of the first interval template (such as the third interval template (such as 450˜499 milliseconds)) and the R-wave interval difference values 320 of the electrocardiography signal to obtain a first cumulative distribution function (CDF) 330 corresponding to the first interval template and a second cumulative distribution function 340 corresponding to the electrocardiography signal.

For example, the processing unit 120 may arrange the values of the R-wave interval difference values of the first interval template (such as the third interval template (such as 450˜499 milliseconds)) and the R-wave interval difference values 320 of the electrocardiography signal in ascending order, perform a normalized process on the above values as values between 0 and 1, and perform the cumulative distribution function calculation on the normalized values to obtain the first cumulative distribution function 330 and the second cumulative distribution function 340.

Then, the processing unit 120 may verify the cumulative distribution function 330 and the second cumulative distribution function 340 using a testing method to obtain a verification value. In some embodiments, the above testing method is, for example, a Kolmogorov-Smirnov test (K-S test) method, and the verification value is, for example, a P value (p-value).

Afterward, the processing unit 120 may classify the electrocardiography signal into the first type or the second type according to the verification value P and a threshold value Tp. Furthermore, the processing unit 120 may compare the verification value P with the threshold value Tp to determine whether the verification value P is greater than the threshold value Tp, so as to classify the electrocardiography signal into the first type or the second type.

For example, when the processing unit 120 determines that the verification value P is greater than the threshold value Tp, the processing unit 120 may classify the electrocardiography signal into the first type (the arrhythmia type). When the processing unit 120 determines that the verification value P is not greater than the threshold value Tp (for example, the verification value P being less than the threshold value Tp), the processing unit 120 may classify the electrocardiography signal into the second type (the non-arrhythmia type).

After the processing unit 120 classifies the electrocardiography signal into the first type, the processing unit 120 may classify the electrocardiography signal of the first type (the arrhythmia type) using the second classification model to classify the electrocardiography signal of the first type (the arrhythmia type) into the first type or the second type. Therefore, the accuracy of the type classification of the electrocardiography signal may be effectively increased.

In some embodiments, the processing unit 120 obtains the training data set. For example, the processing unit 120 may obtain the training data set from the database. In addition, the training data set includes, for example, a known electrocardiography signal of a time length, and the known electrocardiography signal may include the first type (the arrhythmic type) and the second type (the non-arrhythmic type). Furthermore, the above time length is, for example, several minutes or hours, but the embodiment of the present invention is not limited thereto. That is, the training data set may be the same as the training data of the previous embodiment.

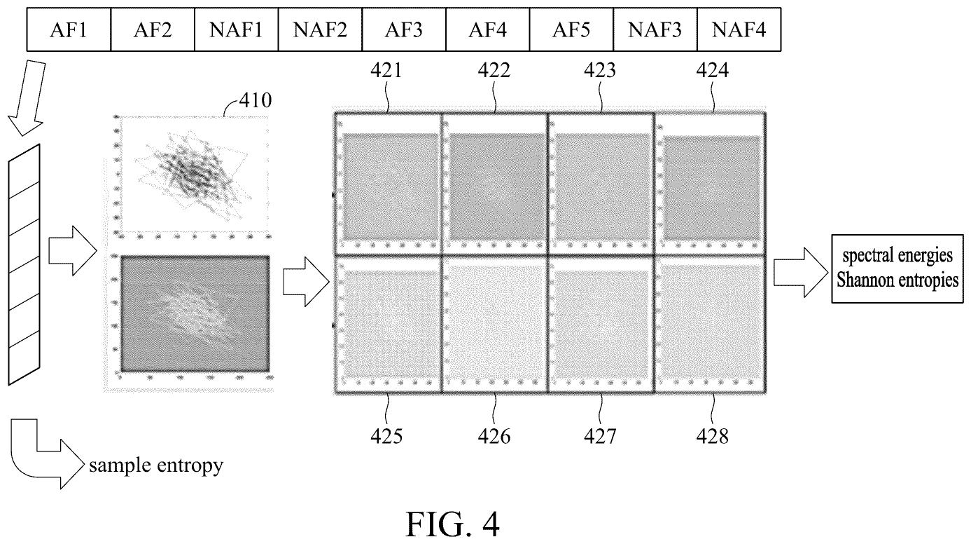

Then, the processing unit 120 may use a predetermined time length to divide the training data set into a plurality of first signal segments AF1˜AF5 and a plurality of second signal segments NAF1˜NAF4, as shown in FIG. 4. In some embodiments, the above predetermined time length is, for example, 30 seconds, but the embodiment of the present invention is not limited thereto. The user may adjust the above predetermined time length according to the requirements thereof. In some embodiments, the type of the first signal segments AF1˜AF5 is different from the type of the second signal segments NAF1˜NAF4. In addition, the first signal segments AF1˜AF5 and the second signal segments NAF1˜NAF4 do not overlap each other.

Afterward, the processing unit 120 may perform an R-wave interval detection on each of the first signal segments AF1˜AF5 and the second signal segments NAF1˜NAF4 to calculate a plurality of R-wave interval segments of each of the first signal segments AF1˜AF5 and the second signal segments NAF1˜NAF4.

Then, the processing unit 120 obtains a sample entropy, a plurality of Shannon entropies and a plurality of spectral energies according to the R-wave interval segments of each of the first signal segments AF1˜AF5 and the second signal segments NAF1˜NAF4.

In some embodiments, the processing unit 120 may rearrange the R-wave interval segments of each of the first signal segments AF1˜AF5 and the second signal segments NAF1˜NAF2 to generate a plurality of sub R-wave interval segment sequences, and calculates the sub R-wave interval segment sequences to obtain the sample entropy.

In the embodiment, the sample entropy may be calculated using equation (1), and equation (1) is as follows.

$$SampEn ( m , r ) = - log ( A / B ) , \qquad ( 1 )$$

wherein SampEn(m, r) is the sample entropy, m represents the number of data points to be compared, usually set to 2, r represents the allowable difference value when comparing two m-dimensional sub sequences, also called the threshold value, A is the probability that the (m+1)-dimensional sub sequences are similar, and B is the probability that m-dimensional sub sequences are similar.

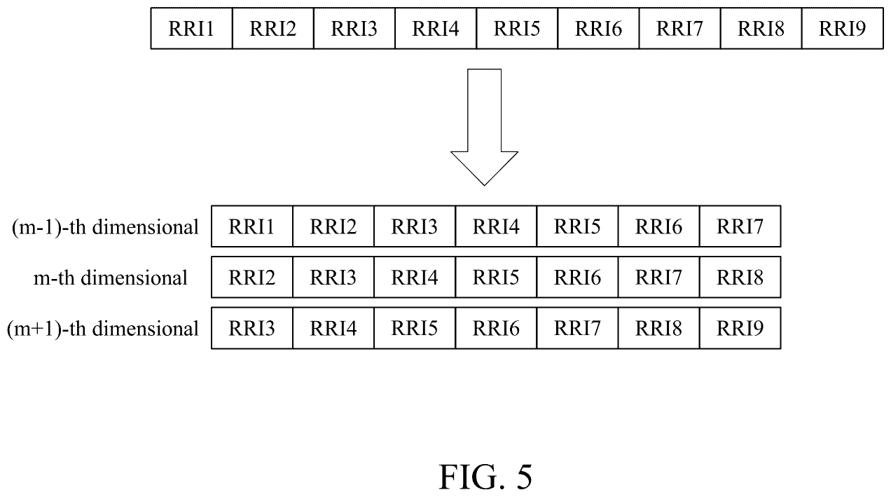

For example, assume that the first signal segment AF1 is taken as an example, the first signal segment AF1 includes the R-wave interval segments RRI1, RRI2, RRI3, RRI4, RRI5, RRI6, RRI7, RRI8 and RRI9, m=2, r=60 milliseconds. The processing unit 120 may rearrange the R-wave interval segments RRI1, RRI2, RRI3, RRI4, RRI5, RRI6, RRI7, RRI8 and RRI9 included in the first signal segment AF1 according to m=2 to generate the sub R-wave interval segment sequences, such as (m−1)-th dimensional sub R-wave interval segment sequence, m-th dimensional sub R-wave interval segment sequence and (m+1)-th dimensional sub R-wave interval segment sequence, as shown in FIG. 5.

In FIG. 5, the (m−1)-th dimensional sub R-wave interval segment sequence may include the R-wave interval segments RRI1, RRI2, RRI3, RRI4, RRI5, RRI6 and RRI7, the m-th dimensional sub R-wave interval segment sequence may include the R-wave interval segments RRI2, RRI3, RRI4, RRI5, RRI6, RRI7 and RRI8, and the (m+1)-th dimensional sub R-wave interval segment sequence R may include the R-wave interval segments RRI3, RRI4, RRI5, RRI6, RRI7, RRI8 and RRI9.

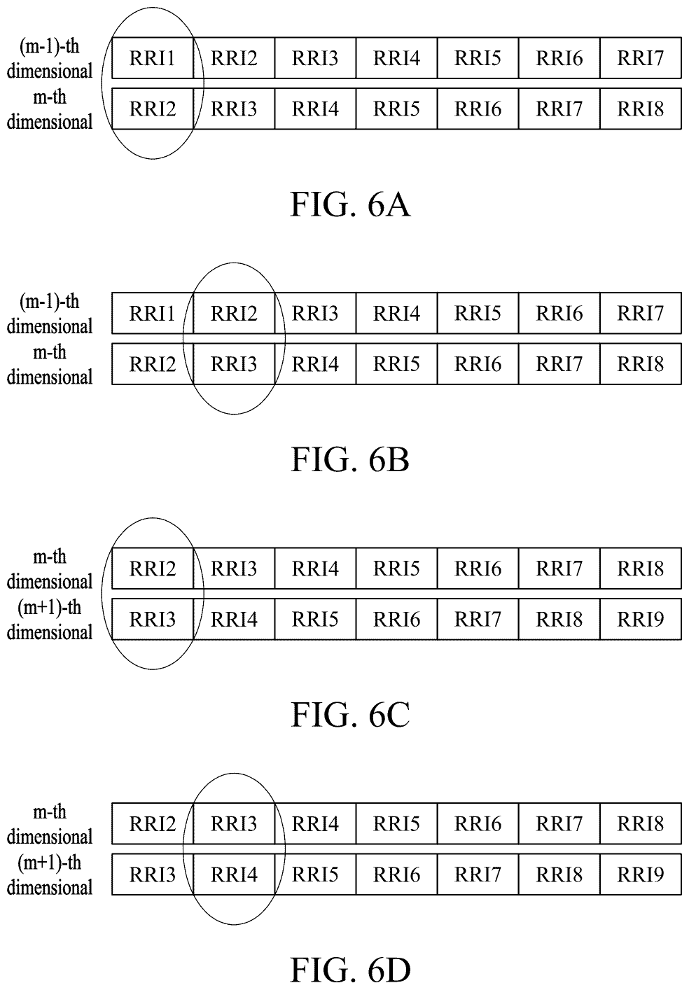

Then, the processing unit 120 may regard the R-wave interval segment RRI1 of the (m−1)-th dimensional sub R-wave interval segment sequence and the R-wave interval segment RRI2 of the m-th dimensional sub R-wave interval segment sequence as a first coordinate point (X1,Y1), and regard the R-wave interval segments RRI2-RRI7 of the (m−1)-th dimensional sub R-wave interval segment sequence and the R-wave interval segments RRI3-RRI8 of the m-th dimensional sub R-wave interval segment sequence as other coordinate points, as shown in FIG. 6A.

Afterward, the processing unit 120 may calculate the distances (such as the Euclidean distances) between the first coordinate point (X1,Y1) and the other 6 coordinate points, such as 6 distance values. Then, the processing unit 120 may compare these 6 distance values with a threshold r (such as 60 milliseconds) to calculate the number of these 6 distance values that are less than the threshold value r. Afterward, if there are 4 distance values less than the threshold r, the processing unit 120 may set A=A+4=0+4=4.

Then, the processing unit 120 may regard the R-wave interval segment RRI2 of the (m−1)-th dimensional sub R-wave interval segment sequence and the R-wave interval segment RRI3 of the m-th dimensional sub R-wave interval segment sequence as a second coordinate point (X2,Y2), and regard the R-wave interval segments RRI1 and RRI3-RRI7 of the (m−1)-th dimensional sub R-wave interval segment sequence and the R-wave interval segments RRI2 and RRI4-RRI8 of the m-th dimensional sub R-wave interval segment sequence as other coordinate points, as shown in FIG. 6B.

Afterward, the processing unit 120 may calculate the distances (such as the Euclidean distances) between the second coordinate point (X2,Y2) and the other 6 coordinate points, such as 6 distance values. Then, the processing unit 120 may compare these 6 distance values with the threshold r (such as 60 milliseconds) to calculate the number of these 6 distance values that are less than the threshold value r. Afterward, if there are 2 distance values less than the threshold r, the processing unit 120 may set A=A+2=4+2=6. In addition, the calculation of the rest of the A values may be deduced by analogy.

Then, the processing unit 120 may regard the R-wave interval segment RRI2 of the m-th dimensional sub R-wave interval segment sequence and the R-wave interval segment RRI3 of the (m+1)-th dimensional sub R-wave interval segment sequence as a first coordinate point (X1,Y1), and regard the R-wave interval segments RRI3-RRI8 of the m-th dimensional sub R-wave interval segment sequence and the R-wave interval segments RRI4-RRI9 of (m+1)-th dimensional sub R-wave interval segment sequence as other coordinate points, as shown in FIG. 6C.

Afterward, the processing unit 120 may calculate the distances (such as the Euclidean distances) between the first coordinate point (X1,Y1) and the other 6 coordinate points, such as 6 distance values. Then, the processing unit 120 may compare these 6 distance values with the threshold r (such as 60 milliseconds) to calculate the number of these 6 distance values that are less than the threshold value r. Afterward, if there are 3 distance values less than the threshold r the processing unit 120 may set B=B+3=0+3=3.

Then, the processing unit 120 may regard the R-wave interval segment RRI3 of the m-th dimensional sub R-wave interval segment sequence and the R-wave interval segment RRI4 of the (m+1)-th dimensional sub R-wave interval segment sequence as a second coordinate point (X2,Y2), and regard the R-wave interval segments RRI2 and RRI4-RRI8 of the m-th dimensional sub R-wave interval segment sequence and the R-wave interval segments RRI3 and RRI5-RRI9 of the (m+1)-th dimensional sub R-wave interval segment sequence as other coordinate points, as shown in FIG. 6D.

Afterward, the processing unit 120 may calculate the distances (such as the Euclidean distances) between the second coordinate point (X2,Y2) and the other 6 coordinate points, such as 6 distance values. Then, the processing unit 120 may compare these 6 distance values with the threshold r (such as 60 milliseconds) to calculate the number of these 6 distance values that are less than the threshold value r. Afterward, if there are 5 distance values less than the threshold r, the processing unit 120 may set B=B+5=3+5=8. In addition, the calculation and the accumulation manner of the rest of the B values may be deduced by analogy. Then, the processing unit 120 may substitute the A value and the B value into equation (1) to calculate the sample entropy. The calculation of sample entropies of the rest of the first signal segments AF2˜AF5 and the second signal segments NAF1˜NAF4 may refer to the description of the calculation of the sample entropy of the first signal segment AF1, and the description thereof is not repeated herein.

In addition, the processing unit 120 may generate a Poincaré plot 410 according to the R-wave interval segments of each of the first signal segments AF1˜AF5 and the second signal segments NAF1˜NAF4, as shown in FIG. 4. In the embodiment, the Poincaré plot is drawn by the heart rate difference values (such as the R-wave interval difference values), wherein the X-axis is N—the heart rate difference value in the time sequence, and Y-axis is (N−1)-th heart rate difference value in the time sequence.

Then, the processing unit 120 may perform a discrete wavelet transform (DWT) and a second-order decomposition on the Poincaré plot a plurality of wavelet transformation diagrams 421, 422, 423, 424, 425, 426, 427 and 428. For example, the processing unit 120 may perform a first-order decomposition on the Poincaré plot to obtain the wavelet transformation diagrams 421, 422, 423 and 424, and then perform the second-order composition on the wavelet transformation diagram 421 to obtain the wavelet transformation diagrams 425, 426, 427 and 428.

Afterward, the processing unit 120 may calculate the Shannon entropies and the spectral energies according to the wavelet transformation diagrams 421, 422, 423, 424, 425, 426, 427 and 428. That is, the processing unit 120 may calculate each of the wavelet transformation diagrams 421, 422, 423, 424, 425, 426, 427 and 428 to obtain the corresponding Shannon entropies and the corresponding spectral energies. In some embodiments, the Shannon entropy may be calculated using equation (2), and equation (2) is as follows.

$$Entropy = - Σ P ( x ) * log 2 ( P ( x ) ) , \qquad ( 2 )$$

wherein Entropy represent the Shannon entropy, and P(x) is the wavelet coefficient probability of the Poincaré plot using the discrete wavelet transform.

In some embodiments, the spectral energy may be calculated using equation (3), and equation (3) is as follows.

$$SE = {\sum }_{n = 1}^{N} {\vert X [ n ] \vert}^{2} , \qquad ( 3 )$$

wherein SE represent the spectral energy, N is the total number of wavelet coefficients, and X[n] is the n-th wavelet coefficient value.

Furthermore, the processing unit 120 may further perform a normalization process on the Shannon entropies and the spectral energies to obtain the normalized Shannon entropies and the normalized spectral energies. Therefore, the processing unit 120 may obtain 33 features (i.e., 1 sample entropy, 16 Shannon entropies and 16) of each of the first signal segments AF1˜AF5 and the second signal segments NAF1˜NAF4.

Afterward, the processing unit 120 may a feature screening on the sample entropy, the Shannon entropies and the spectral energies corresponding to each of the first signal segments AF1˜AF5 and the second signal segments NAF1˜NAF4 using a feature screening method to obtain a plurality of training features corresponding to each of the first signal segments AF1˜AF5 and the second signal segments NAF1˜NAF4. In some embodiments, the above feature screening method may include a wrapper method, an intrinsic method, an implicit method and a filter method.

For example, assume that the wrapper method is taken as an example, the processing unit 120 may separately train the 33 features to select the feature with the lowest loss among the 33 features, such as the first feature. Then, the processing unit 120 may separately train the first feature plus each of the remaining 32 features and use, for example, a 10-fold cross-validation method to select a combination in which the model loss is lower than the previous model loss in these combinations (the addition of the two features), such as the first feature and the third feature.

Afterward, the processing unit 120 may separately train the combination of the first feature and the third feature plus each of the remaining 31 features and use, for example the 10-fold cross-validation method to select a combination in which the model loss is lower than the previous model loss in these combinations (the addition of the three features), such as the first feature, the third feature and the sixth feature. Then, the processing unit 120 may use the first feature, the third feature and the sixth feature as candidates and perform subsequent tests. Until the processing unit 120 determines that the current model loss no longer decreases, the processing unit 120 may stop feature screening, and use the combination of features at this time as a training feature.

In addition, the number of training features is, for example, less than 33 features (i.e., 1 sample entropy, 16 Shannon entropies and 16 spectral energies). In the embodiment, the processing unit 120 obtains, for example, 15 features, but the embodiment of the present invention is not limited thereto. Furthermore, the above 10-fold cross-validation method is an implementation example of the present invention, but the embodiment of the present invention is not limited thereto. In other embodiments, the user may use other k-fold cross-validation method, and the same effect or similar effect may also be achieved.

Afterward, the processing unit 120 may generate the second classification model according to the training features (such as 15 training features) and the training data set. Then, the processing unit 120 may classify the electrocardiography signal of the first type into the first type or the second type according to the second classification model. That is, the processing unit 120 may perform a similarity comparison on the electrocardiography signal of the first type and the second classification model to generate a probability value, and classify the electrocardiography signal of the first type into the first type or the second type according to the probability value and a threshold value. For example, when the above probability value is greater than the threshold value, the processing unit 120 may classify the electrocardiography signal into the first type. When the above probability value is less than the threshold value, the processing unit 120 may classify the electrocardiography signal into the second type.

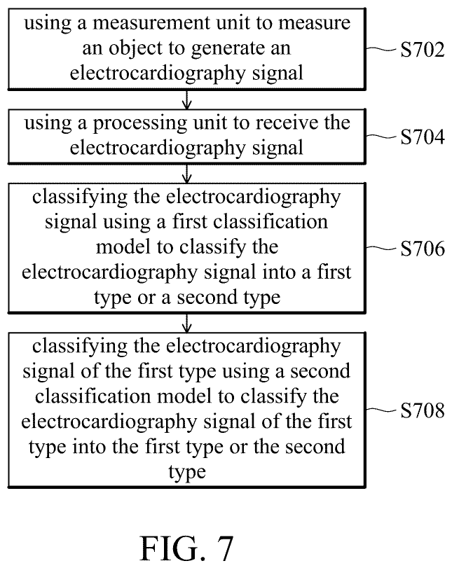

FIG. 7 is a flowchart of an operation method of an electrocardiography processing device according to an embodiment of the present invention. In step S702, the method involves using a measurement unit to measure an object to generate an electrocardiography signal. In step S704, the method involves using a processing unit to receive the electrocardiography signal. In step S706, the method involves classifying the electrocardiography signal using a first classification model to classify the electrocardiography signal into a first type or a second type.

In step S708, the method involves classifying the electrocardiography signal of the first type using a second classification model to classify the electrocardiography signal of the first type into the first type or the second type. In the embodiment, the first type is, for example, the arrhythmic type, and the second type is, for example, the non-arrhythmic type.

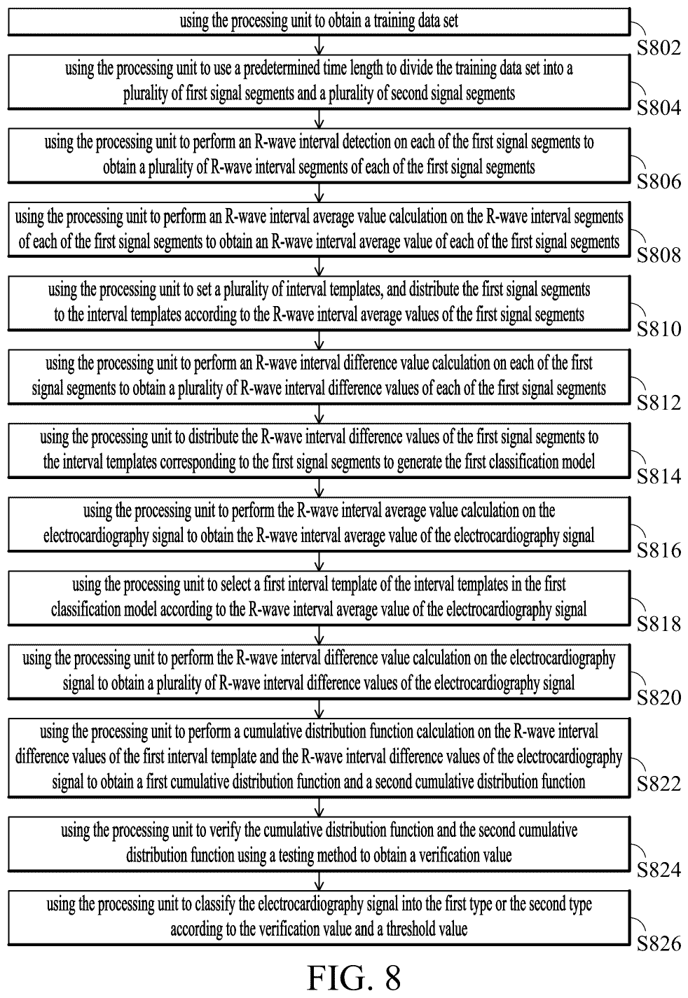

FIG. 8 is a detailed flowchart of step S706 in FIG. 7. In step S802, the method involves using the processing unit to obtain a training data set. In step S804, the method involves using the processing unit to use a predetermined time length to divide the training data set into a plurality of first signal segments and a plurality of second signal segments. In step S806, the method involves using the processing unit to perform an R-wave interval detection on each of the first signal segments to obtain a plurality of R-wave interval segments of each of the first signal segments.

In step S808, the method involves using the processing unit to perform an R-wave interval average value calculation on the R-wave interval segments of each of the first signal segments to obtain an R-wave interval average value of each of the first signal segments. In step S810, the method involves using the processing unit to set a plurality of interval templates, and distribute the first signal segments to the interval templates according to the R-wave interval average values of the first signal segments.

In step S812, the method involves using the processing unit to perform an R-wave interval difference value calculation on each of the first signal segments to obtain a plurality of R-wave interval difference values of each of the first signal segments. In step S814, the method involves using the processing unit to distribute the R-wave interval difference values of the first signal segments to the interval templates corresponding to the first signal segments to generate the first classification model.

In step S816, the method involves using the processing unit to perform the R-wave interval average value calculation on the electrocardiography signal to obtain the R-wave interval average value of the electrocardiography signal. In step S818, the method involves using the processing unit to select a first interval template of the interval templates in the first classification model according to the R-wave interval average value of the electrocardiography signal. In step S820, the method involves using the processing unit to perform the R-wave interval difference value calculation on the electrocardiography signal to obtain a plurality of R-wave interval difference values of the electrocardiography signal.

In step S822, the method involves using the processing unit to perform a cumulative distribution function calculation on the R-wave interval difference values of the first interval template and the R-wave interval difference values of the electrocardiography signal to obtain a first cumulative distribution function and a second cumulative distribution function. In step S824, the method involves using the processing unit to verify the cumulative distribution function and the second cumulative distribution function using a testing method to obtain a verification value. In step S826, the method involves using the processing unit to classify the electrocardiography signal into the first type or the second type according to the verification value and a threshold value.

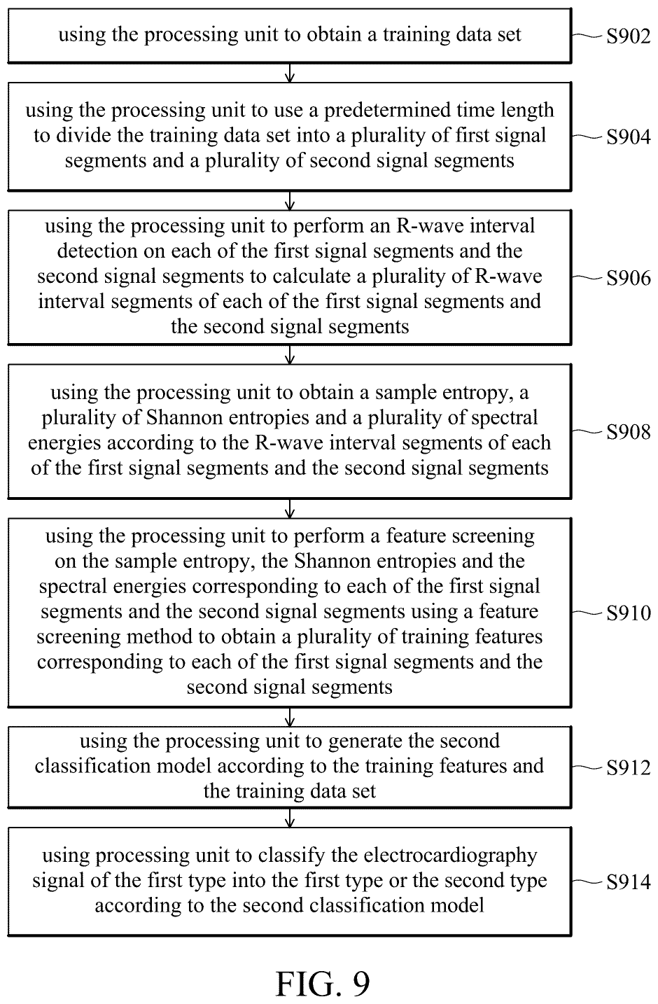

FIG. 9 is a detailed flowchart of step S708 in FIG. 7. In step S902, the method involves using the processing unit to obtain a training data set. In step S904, the method involves using the processing unit to use a predetermined time length to divide the training data set into a plurality of first signal segments and a plurality of second signal segments. In step S906, the method involves using the processing unit to perform an R-wave interval detection on each of the first signal segments and the second signal segments to calculate a plurality of R-wave interval segments of each of the first signal segments and the second signal segments.

In step S908, the method involves using the processing unit to obtain a sample entropy, a plurality of Shannon entropies and a plurality of spectral energies according to the R-wave interval segments of each of the first signal segments and the second signal segments. In step S910, the method involves using the processing unit to perform a feature screening on the sample entropy, the Shannon entropies and the spectral energies corresponding to each of the first signal segments and the second signal segments using a feature screening method to obtain a plurality of training features corresponding to each of the first signal segments and the second signal segments. In step S912, the method involves using the processing unit to generate the second classification model according to the training features and the training data set. In step S914, the method involves using processing unit to classify the electrocardiography signal of the first type into the first type or the second type according to the second classification model.

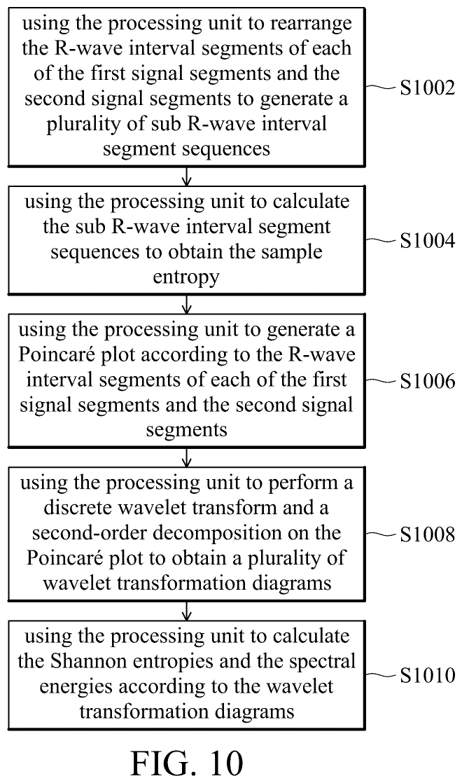

FIG. 10 is a detailed flowchart of step S908 in FIG. 9. In step S1002, the method involves using the processing unit to rearrange the R-wave interval segments of each of the first signal segments and the second signal segments to generate a plurality of sub R-wave interval segment sequences. In step S1004, the method involves using the processing unit to calculate the sub R-wave interval segment sequences to obtain the sample entropy.

In step S1006, the method involves using the processing unit to generate a Poincaré plot according to the R-wave interval segments of each of the first signal segments and the second signal segments. In step S1008, the method involves using the processing unit to perform a discrete wavelet transform and a second-order decomposition on the Poincaré plot to obtain a plurality of wavelet transformation diagrams. In step S1010, the method involves using the processing unit to calculate the Shannon entropies and the spectral energies according to the wavelet transformation diagrams.

In summary, according to the electrocardiography processing device disclosed by the embodiment of the present invention, the processing unit receives the electrocardiography signal generated by the measurement unit, classifies the electrocardiography signal using the first classification model to classify the electrocardiography signal into the first type or the second type, and classifies the electrocardiography signal of the first type using the second classification model to classify the electrocardiography signal of the first type into the first type or the second type. Therefore, the accuracy of the type classification of the electrocardiography signal may be effectively increased.

While the present invention has been described by way of example and in terms of the preferred embodiments, it should be understood that the present invention is not limited to the disclosed embodiments. On the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. An electrocardiography processing device, comprising:
a measurement unit, configured to measure an object to generate an electrocardiography signal; and
a processing unit, configured to receive the electrocardiography signal, classify the electrocardiography signal using a first classification model to classify the electrocardiography signal into a first type or a second type, and classify the electrocardiography signal of the first type using a second classification model to classify the electrocardiography signal of the first type into the first type or the second type.

2. The electrocardiography processing device as claimed in claim 1, wherein the processing unit obtains a training data set;
the processing unit uses a predetermined time length to divide the training data set into a plurality of first signal segments and a plurality of second signal segments;
the processing unit performs an R-wave interval detection on each of the plurality of first signal segments to obtain a plurality of R-wave interval segments of each of the plurality of first signal segments;
the processing unit performs an R-wave interval average value calculation on the plurality of R-wave interval segments of each of the plurality of first signal segments to obtain an R-wave interval average value of each of the plurality of first signal segments;
the processing unit sets a plurality of interval templates, and distributes the plurality of first signal segments to the plurality of interval templates according to the R-wave interval average values of the plurality of first signal segments;
the processing unit performs an R-wave interval difference value calculation on each of the plurality of first signal segments to obtain a plurality of R-wave interval difference values of each of the plurality of first signal segments;
the processing unit distributes the plurality of R-wave interval difference values of the plurality of first signal segments to the plurality of interval templates corresponding to the plurality of first signal segments to generate the first classification model.

3. The electrocardiography processing device as claimed in claim 2, wherein the processing unit performs the R-wave interval average value calculation on the electrocardiography signal to obtain the R-wave interval average value of the electrocardiography signal;
the processing unit selects a first interval template of the plurality of interval templates in the first classification model according to the R-wave interval average value of the electrocardiography signal; and
the processing unit performs the R-wave interval difference value calculation on the electrocardiography signal to obtain a plurality of R-wave interval difference values of the electrocardiography signal.

4. The electrocardiography processing device as claimed in claim 3, wherein the processing unit performs a cumulative distribution function calculation on the R-wave interval difference values of the first interval template and the plurality of R-wave interval difference values of the electrocardiography signal to obtain a first cumulative distribution function and a second cumulative distribution function;
the processing unit verifies the cumulative distribution function and the second cumulative distribution function using a testing method to obtain a verification value; and
the processing unit classifies the electrocardiography signal into the first type or the second type according to the verification value and a threshold value.

5. The electrocardiography processing device as claimed in claim 4, wherein the testing method is a Kolmogorov-Smirnov test method.

6. The electrocardiography processing device as claimed in claim 1, wherein the processing unit obtains a training data set;
the processing unit uses a predetermined time length to divide the training data set into a plurality of first signal segments and a plurality of second signal segments;
the processing unit performs an R-wave interval detection on each of the plurality of first signal segments and the plurality of second signal segments to calculate a plurality of R-wave interval segments of each of the plurality of first signal segments and the plurality of second signal segments;
the processing unit obtains a sample entropy, a plurality of Shannon entropies and a plurality of spectral energies according to the plurality of R-wave interval segments of each of the plurality of first signal segments and the plurality of second signal segments;
the processing unit performs a feature screening on the sample entropy, the plurality of Shannon entropies and the plurality of spectral energies corresponding to each of the plurality of first signal segments and the plurality of second signal segments using a feature screening method to obtain a plurality of training features corresponding to each of the plurality of first signal segments and the plurality of second signal segments;
the processing unit generates the second classification model according to the plurality of training features and the training data set; and
the processing unit classifies the electrocardiography signal of the first type into the first type or the second type according to the second classification model.

7. The electrocardiography processing device as claimed in claim 6, wherein the processing unit rearranges the plurality of R-wave interval segments of each of the plurality of first signal segments and the plurality of second signal segments to generate a plurality of sub R-wave interval segment sequences, and calculates the plurality of sub R-wave interval segment sequences to obtain the sample entropy.

8. The electrocardiography processing device as claimed in claim 6, wherein the processing unit generates a Poincaré plot according to the plurality of R-wave interval segments of each of the plurality of first signal segments and the plurality of second signal segments;
the processing unit performs a discrete wavelet transform and a second-order decomposition on the Poincaré plot to obtain a plurality of wavelet transformation diagrams;
the processing unit calculates the plurality of Shannon entropies and the plurality of spectral energies according to the plurality of wavelet transformation diagrams.

9. The electrocardiography processing device as claimed in claim 8, wherein the processing unit further performs a normalization process on the plurality of Shannon entropies and the plurality of spectral energies.

10. The electrocardiography processing device as claimed in claim 6, wherein the feature screening method comprises a wrapper method, an intrinsic method, an implicit method and a filter method.
